(19) United States
(12) Patent Application Publication
(10) Pub. No.: US 2009/0007924 A1
(43) Pub. Date: Jan. 8, 2009

(54) ARBITER SYSTEM AND METHOD OF COMPUTERIZED MEDICAL DIAGNOSIS AND ADVICE

(76) Inventor: Edwin C. Iliff, La Jolla, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 12/113,132

(22) Filed: Apr. 30, 2008

Related U.S. Application Data
(60) Provisional application No. 60/915,047, filed on Apr. 30, 2007.

(51) Int. Cl. A61B 5/00; A61B 19/00

(52) U.S. Cl. 128/898; 600/300

(57) ABSTRACT

A computerized medical diagnostic system and method is described. A computer storage stores a list of candidate disease objects, where each disease object is associated with one or more questions. A computing device is in data communication with the computer storage, and executes instructions associated with an arbiter object. The arbiter object utilizes at least one of multiple evaluation strategies that determine the selection of a next best question to ask of a patient. The disease objects can be separated into a first class that is allowed to vote for the next best question which is to be asked of the patient or into a second class that is not allowed to vote for the next best question. The arbiter object can determine when a next evaluation strategy of the multiple evaluation strategies is to be started.

### CROSS-REFERENCE TO RELATED APPLICATIONS

This application claims the benefit of U.S. Provisional Application No. 60/915047, filed Apr. 30, 2007, which is hereby incorporated by reference in its entirety.

### BACKGROUND

1. Field of the Invention

This invention relates to computerized medical diagnosis and advice, and more particularly to allowing different diagnostic or evaluation strategies to be used.

2. Description of the Related Technology

Known medical diagnosis methods have a single method of diagnosis.

### SUMMARY OF CERTAIN INVENTIVE EMBODIMENTS

In one embodiment there is a computerized arbiter method utilized during an evaluation session in a medical diagnostic system having a computing device, the method comprising asking general questions associated with a list of candidate diseases of a patient using a high level mode of inquiry by use of a user interface associated with a computing device; selecting a set of most likely diseases based on the responses to the general questions; asking questions focused on the set of most likely diseases using a middle level mode of inquiry; selecting a most likely disease based on the responses to the questions from the middle level of inquiry; and asking questions focused on the most likely disease using a low level mode of inquiry, wherein a sequence of questions corresponds to one of a plurality of evaluation strategies, wherein the set of most likely diseases is divided into one class that is allowed to vote for the next best question which is to be asked of the patient or into another class that is not allowed to vote for the next best question, wherein the diseases that are in the class that cannot vote for the next best question add a weight to a disease score, the weight corresponding to a response for a question asked by another disease, and wherein questions are asked until a goal of the evaluation session has been reached.

One of the evaluation strategies may be intent modulation which eliminates the later stages of urgent diseases from the list of candidate diseases. The responses to the questions may be stored in a patient electronic medical record and may be used to establish patient health items (PHIs), and wherein each candidate disease may be associated with one or more PHIs, and each PHI may be associated with one or more questions. One of the evaluation strategies may be mean democratic sine which determines the next best question by a voting process wherein the sine status of a PHI and disease pair is factored into the voting strength of the diseases. One of the evaluation strategies may be a sequential synergy strategy which gives more voting strength of priority to those PHIs that complete or nearly complete a sequential synergy. The diseases that are in the class that are allowed to vote for the next best question may add a weight to a disease score. The next best question may be the question that advances the evaluation session to reach a correct diagnosis at the earliest point in time with the fewest number of questions. The number of questions asked of the patient may be reduced based on use of the middle level of inquiry where the class that is not allowed to vote for the next best question does not contribute potential questions to be asked of the patient. The questions corresponding to late stage PHIs of urgent diseases may be asked first so as to diagnose or exclude those diseases that have a limited therapeutic window of opportunity. A particular one of the plurality of evaluation strategies can be changed responsive to a clinical situation of the patient. A particular one of the plurality of evaluation strategies may be selected depending upon a past medical history of the patient as stored in the patient electronic medical record. A particular one of the plurality of evaluation strategies may be selected depending upon the patient's previous responses in a consultation. The method may additionally comprise selecting from the questions voted on by the diseases in diagnostic consideration. The method may not permit diseases of the class not allowed to vote of the set of most likely diseases to suggest questions to ask of the patient during the evaluation session.

In another embodiment there is a computerized medical diagnostic system, the system comprising a computer storage storing a list of candidate diseases, each candidate disease associated with one or more questions; a computing device in data communication with the computer storage, the computing device performing software instructions to ask general questions associated with the candidate diseases of a patient using a high level mode of inquiry; select a set of most likely diseases based on the responses to the general questions; ask questions focused on the set of most likely diseases using a middle level mode of inquiry; select a most likely disease based on the responses to the questions from the middle level of inquiry; and ask questions focused on the most likely disease using a low level mode of inquiry, wherein the set of most likely diseases may be separated into a first class that is allowed to vote for the next best question which is to be asked of the patient or into a second class that is not allowed to vote for the next best question, wherein the diseases that are in the second class add a weight to a disease score, the weight corresponding to a response for a question asked by another disease, and wherein questions are asked until a goal of the evaluation session has been reached.

The responses to the questions may be stored in a patient electronic medical record and may be used to establish patient health items (PHIs). The diseases in the list of candidate diseases may be separated into the first class or the second class based on at least the PHIs. The diseases in the first class may add a weight corresponding to a response for a question asked by another disease. A sequence of questions may correspond to one of a plurality of evaluation strategies. The separation of the diseases into the first class and the second class may be dynamic and may be based in part on a voting strength of each disease in the list of candidate diseases. The voting strength of a particular disease may be related to the changing probability that the particular disease is the diagnosis for the patient. The voting strength of a particular disease may be dependent upon the number of PHIs the patient has of the particular disease. The voting strength may depend upon aspects of the PHI being established for the patient. A particular disease may be dynamically transferred between the first and second classes upon reaching or exceeding a threshold. The system may not permit diseases of the class not allowed to vote to suggest questions to ask of the patient during an evaluation session based at least in part on the disease score. Each candidate disease may be associated with one or more patient health items (PHIs), and each PHI may be associated with one or more questions. The computing device may additionally perform software instructions to check the patient electronic medical record for responses to questions or PHIs prior to asking questions of the patient.

In another embodiment there is a computerized medical diagnostic system, the system comprising a computer storage storing a list of disease objects, each disease object associated with one or more questions; and a computing device in data communication with the computer storage, the computing device executing instructions associated with an arbiter object, wherein the arbiter object, in conjunction with a plurality of evaluation strategies, determines the selection of a next best question to ask of a patient.

The arbiter object may determine when the next evaluation strategy of the plurality of evaluation strategies is to be started. The determination of when the next evaluation strategy may be to be started is based on a rule set. The determination of when the next evaluation strategy may be to be started depends on the completion of the current evaluation strategy. The system may additionally comprise a patient ombudsman object that interfaces with the arbiter object and may suggest one or more general questions, wherein answers to the general questions causes a decrease in the number of questions asked of the patient. An evaluation strategy may be intent modulation in which the late stage of urgent diseases are established or ruled out before proceeding to other diseases. An evaluation strategy may be intent modulation in which critical curve patient health items (PHIs) of urgent diseases are established after evaluating late stage symptoms. An evaluation strategy may comprise excluding or establishing serious diseases before diagnosing other diseases. The evaluation strategies can be changed as often as after every question asked of the patient. Certain PHIs of a disease may be designated as late stage PHIs of a disease. Certain PHIs of a disease may be designated as critical curve PHIs. The arbiter object may not permit disease objects of a class not allowed to vote from the list of disease objects to suggest questions to ask of the patient during an evaluation session. Each disease object may be associated with one or more PHIs, and each PHI is associated with one or more questions. The arbiter object may interface the disease objects with the patient. Selected ones of the disease objects may suggest questions to ask of the patient, and wherein the arbiter object may select the next best question to ask the patient based at least on a voting strength of the selected disease objects. The arbiter object may select the next best question to ask the patient additionally based at least on a weight of the question, a sine status of a patient health item associated with the question, the diseases in diagnostic consideration, and data in an electronic medical record of the patient. The system may additionally comprise an interface in data communication with an output device to ask questions of the patient and with an input device to receive responses from the patient. The arbiter object can change the axis of inquiry based on certain criteria.

In another embodiment there is a computerized arbiter method associated with an evaluation session in a medical diagnostic system, the method comprising providing a plurality of modes of inquiry, each mode including at least one evaluation strategy, including one mode of inquiry wherein a plurality of disease objects are separated into a first class that is allowed to vote for the next best question which is to be asked of a patient or into a second class that is not allowed to vote for the next best question to be asked; and asking the next best question of the patient.

The method may additionally comprise asking general questions of the patient associated with a list of diseases using a high level mode of inquiry; selecting a set of most likely diseases based on the responses to the general questions; asking questions focused on the set of most likely diseases using a middle level mode of inquiry; selecting a most likely disease based on the responses to the questions from the middle level of inquiry; and asking questions focused on the most likely disease using a low level mode of inquiry, wherein the evaluation strategies include at least one diagnostic strategy, wherein each of the diseases that has not been excluded from diagnostic consideration adds a weight to a disease score based on a response to each question asked, and wherein questions are asked until a goal of the evaluation session has been reached. The evaluation strategies may include a non-diagnostic strategy. The method may not permit diseases of a non-voting class from the set of most likely diseases to suggest questions to ask of the patient during the evaluation session. A disease may be excluded if weights for the patient health items for which questions have not been asked yet and weights with associated synergies cannot cause the disease score to reach or exceed a diagnostic threshold.

In yet another embodiment there is a computerized medical diagnostic system, the system comprising a computer storage storing a list of candidate disease objects, each disease object associated with one or more questions; and a computing device in data communication with the computer storage, the computing device executing instructions associated with an arbiter object, wherein the arbiter object utilizes at least one of a plurality of evaluation strategies that help determine the selection of a next best question to ask of a patient, wherein the disease objects are separated into a first class that is allowed to vote for the next best question which is to be asked of the patient or into a second class that is not allowed to vote for the next best question. The arbiter object may determine when a next evaluation strategy of the plurality of evaluation strategies is to be started.

### BRIEF DESCRIPTION OF THE DRAWINGS

FIG. 1a is a block diagram of an embodiment of an example configuration of a medical diagnostic and treatment advice system.

FIG. 1b is a flowchart of an embodiment of an arbiter process performed by the medical diagnostic and advice system.

FIG. 2 is a flowchart of an embodiment of an initialize new arbiter process shown in FIG. 1b.

FIG. 3 is a flowchart of an embodiment of a select strategy process shown in FIG. 1b.

FIG. 4 is a flowchart of an embodiment of a select patient health item (PHI) process shown in FIG. 1b.

FIG. 5 is a flowchart of an embodiment of an intent modulation strategy process shown in FIG. 4.

FIG. 6 is a flowchart of an embodiment of a resolve PHI process shown in FIG. 1b.

FIG. 7 is a flowchart of an embodiment of an urgent late stage symptoms process shown in FIG. 6.

FIG. 8 is a flowchart of an embodiment of an update disease object lists process shown in FIG. 1b.

FIG. 9 is a flowchart of an embodiment of a mean democratic sine strategy process shown in FIG. 4.

FIG. 10 is a flowchart of an embodiment of a sequential synergy strategy process shown in FIG. 4.

FIG. 11 is a flowchart of an embodiment of a mean democratic strategy process shown in FIG. 4.

FIG. 12a is a diagram of an embodiment of a simplified example configuration of disease objects in a medical diagnostic and treatment advice system interacting with a patient.

FIG. 12b is a diagram of an embodiment of an example configuration of arbiter components in a medical diagnostic and treatment advice system.

FIG. 13 is a diagram of an embodiment of an example configuration of arbiter components operating in a horizontal axis of inquiry mode.

FIG. 14 is a diagram of an embodiment of an example configuration of arbiter components operating in a diagonal axis of inquiry mode.

FIG. 15 is a diagram of an embodiment of an example configuration of arbiter components operating in a vertical axis of inquiry mode.

FIG. 16 is a block diagram of another embodiment of an exemplary configuration of components of a medical diagnostic and treatment advice system.

### DETAILED DESCRIPTION OF CERTAIN PREFERRED EMBODIMENTS

The following detailed description of certain embodiments presents various descriptions of specific embodiments of the invention. However, the invention can be embodied in a multitude of different ways.

The terminology used in the description presented herein is not intended to be interpreted in any limited or restrictive manner, simply because it is being utilized in conjunction with a detailed description of certain specific embodiments of the invention. Furthermore, embodiments of the invention may include several novel features, no single one of which is solely responsible for its desirable attributes or which is essential to practicing the embodiments herein described.

The system is comprised of various modules, tools, and applications. As can be appreciated by one of ordinary skill in the art, each of the modules may comprise various sub-routines, procedures, definitional statements and macros. Each of the modules are typically separately compiled and linked into a single executable program. Therefore, the following description of each of the modules is used for convenience to describe the functionality of the preferred system. Thus, the processes that are undergone by each of the modules may be arbitrarily redistributed to one of the other modules, combined together in a single module, or made available in, for example, a shareable dynamic link library.

The system modules, tools, and applications may be written in any programming language such as, for example, C, C++, C#, BASIC, Visual Basic, Pascal, Ada, Java, HTML, XML, or FORTRAN, and executed on an operating system, such as variants of Windows, Macintosh, UNIX, Linux, VxWorks, PalmOS, PocketPC, Symbian, Java-based or other operating system. C, C++, C#, BASIC, Visual Basic, Pascal, Ada, Java, HTML, XML and FORTRAN are industry standard programming languages for which many commercial compilers can be used to create executable code.

The present system and method allows many different modes of inquiry, which are in themselves dependent on the progress of the diagnostic process. Three modes or axes, Horizontal Axis of Inquiry (HAI), Diagonal Axis of Inquiry (DAI) and the Vertical Axis of Inquiry (VAI), permit a List-Based Engine or Arbiter object to vary its focus from the general, e.g., considering many possible diseases to the specific, e.g., considering one disease. In the early stages, the engine knows little about the patient and must ask the best general questions that quickly eliminate a large number of candidate diseases. But after applying the Horizontal Axis of Inquiry (HAI) for a while, if the scores or diagnostic momentum of some diseases reaches a specified level, the engine can then switch to the Diagonal Axis of Inquiry (DAI) to focus the diagnostic process on a subset of diseases and later into the Vertical Axis of Inquiry (VAI) to focus on a single disease, to the momentary exclusion of all other diseases. In addition, within each axis the engine may also employ multiple diagnostic strategies.

The Arbiter object facilitates the evaluation and switching of modes of inquiry based upon evaluation strategies designed to achieve a diagnosis in as few iterative steps as possible, or stated another way, to reach the diagnosis in as few questions as possible. In addition, the system allows the primary diagnosis to be performed by the software equivalent of a world-class expert in the disease that the patient has. In certain embodiments, evaluation strategies can include one or more diagnostic strategies and one or more non-diagnostic strategies. An example of a non-diagnostic strategy is a strategy that excludes the urgent or serious problems and then not pursuing the diagnosis either at all or to do it in a latter (reenter) consultation.

The Arbiter object has the ability to employ multiple diagnostic strategies based upon the purpose or goal of the consultation (e.g., diagnose, rule out worst case diagnoses), the stage of the consultation and the diseases in diagnostic consideration, how sensitive or how thorough the patient wants the evaluation to be, etc. Because of the modular nature of the Arbiter, evaluation strategies can be added and removed, yielding a “best fit” solution to most any given medical diagnostic requirements. The Arbiter object can change strategies or can change the axis of inquiry or can change both.

The Arbiter object is designed to “prune” execution to “least cost” (fewest data required to achieve solution) thereby maximizing the efficiency of the diagnostic process as a whole, and yielding lower costs, as diseases can be caught in earlier stages of progression, reducing the cost of treatment.

### System Overview

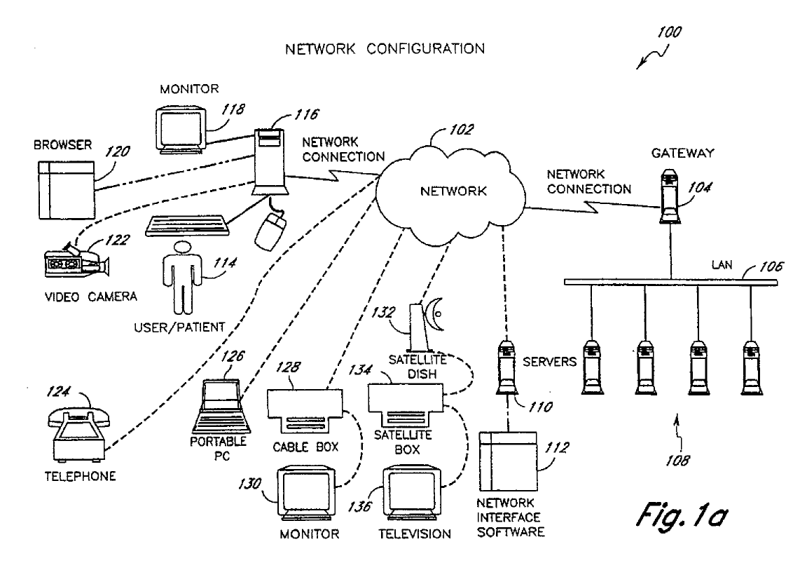

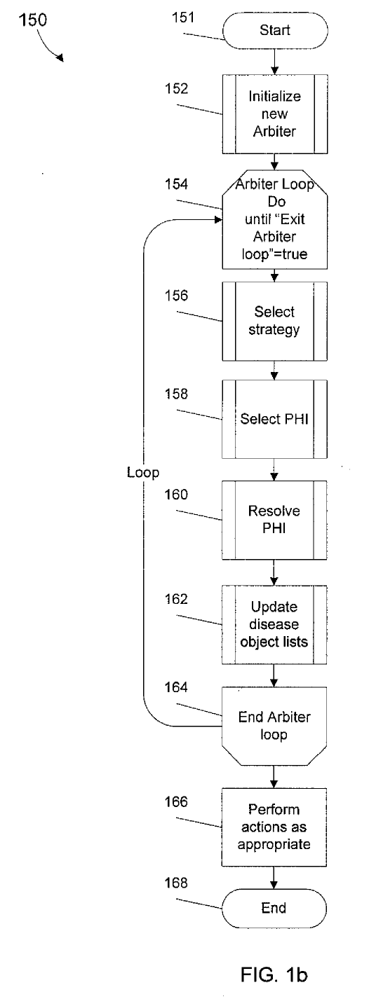

A Medical Diagnostic and Treatment Advice (MDATA) system is a computer system that conducts automated interviews of patients for the purpose of establishing a medical diagnosis. Referring to FIG. 1a, a block diagram of an embodiment of the MDATA system 100 will be described. The MDATA system 100 includes a network “cloud” 102, which may represent a local area network (LAN), a wide area network (WAN), the Internet, or another connection service.

The MDATA programs and databases preferably reside on a group of servers 108 that are preferably interconnected by a LAN 106 and a gateway 104 to the network 102. Alternatively, the MDATA programs and databases reside on a single server 110 that utilizes network interface hardware and software 112.

The network 102 may connect (wired or wirelessly) to a user computer 116 or other computing device, for example, by use of a modem or by use of a network interface card. A user 114 at computer 116 may utilize a browser 120 to remotely access the MDATA programs using a keyboard and/or pointing device and a visual display, such as monitor 118. Alternatively, the browser 120 is not utilized when the MDATA programs are executed in a local mode on computer 116. An interface (not shown), such as a graphical user interface, is used to provide or ask questions of the user 114 (e.g., patient or patient proxy in certain embodiments) and receive answers from the user. A video camera 122 may be optionally connected to the computer 116 to provide visual input, such as visual symptoms. The arbiter method may be realized in a program format to be stored on a computer readable recording medium that includes any kinds of recording devices for storing computer readable data, for example, a CD-ROM, a DVD, a magnetic tape, memory card and a disk, and may also be realized in a carrier wave format (e.g., Internet transmission or Bluetooth transmission).

Various other devices may be used to communicate with the MDATA servers 108/110. If the servers are equipped with voice recognition or DTMF hardware, the user can communicate with the MDATA program by use of a telephone 124. A telephonic embodiment is described in Applicant's application entitled “Computerized Medical Diagnostic and Treatment Advice System,” U.S. Ser. No. 08/176,041, now U.S. Pat. No. 5,660,176, which is hereby incorporated by reference. Other connection devices for communicating with the MDATA servers 108/110 include a portable personal computer or other handheld computing device with a modem or wireless connection interface, a cable interface device 128 connected to a visual display 130, or a satellite dish 132 connected to a satellite receiver 134 and a television 136. Other ways of allowing communication between the user 114 and the MDATA servers 108/110 are envisioned. The MDATA system is further described in U.S. Pat. No. 5,935,060, which is hereby incorporated by reference in its entirety.

### The Arbiter Paradigm

An Arbiter object functions as a computerized patient intermediary between the disease objects which are involved in the process of diagnosing the patient. Software objects include software procedures and functions (methods) and encapsulated data, such as described in U.S. Pat. No. 6,468,210, which is hereby incorporated by reference in its entirety. In addition, it also recommends the best diagnostic strategy to employ depending upon a number of parameters including the diseases in diagnostic consideration, the stage of the consultation, and the goal of the consultation. A goal of the Arbiter may be said to be to find the next best question to ask the patient. In certain embodiments, the next best question is the question that advances a patient evaluation to get a correct diagnosis at the earliest point in time with the fewest number of questions. Working with an automated Medical Diagnostic and Treatment Advice system, the Arbiter represents a process enhancement with a design goal to achieve diagnostic threshold in as few steps as possible.
A goal is to:

Get the right disease (diagnosis)
At the earliest point in time,
With the fewest questions.

### Process Environment Analogy

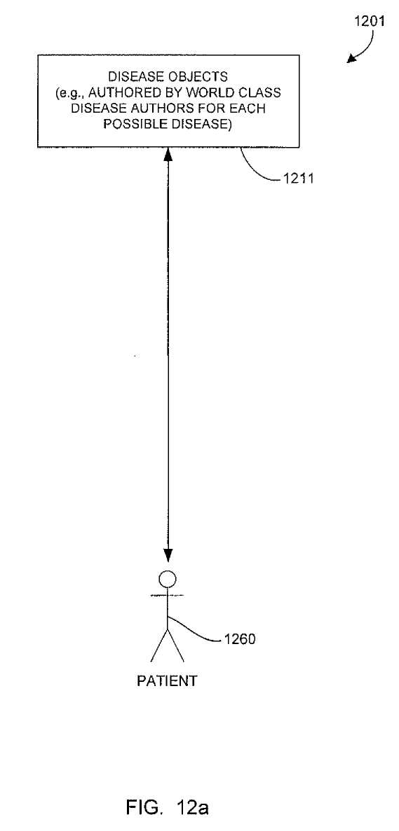

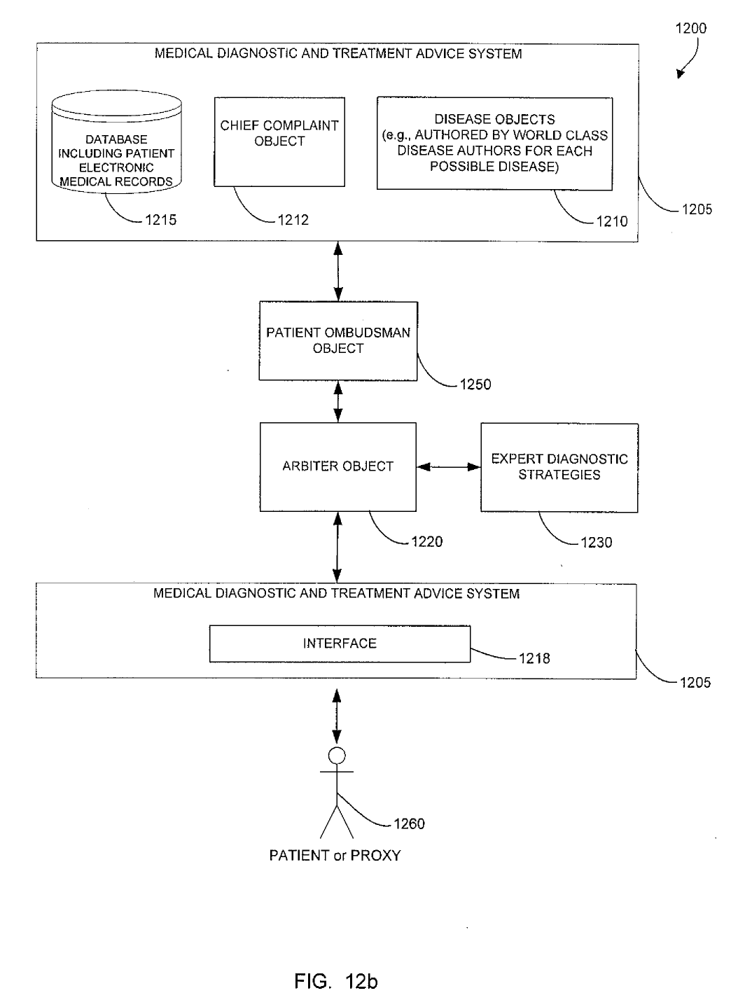

In a process environment analogy, there will be an auditorium with one hundred expert headache neurologists, for example. Each will be a world class expert in one disease causing headache. In certain embodiments, during the interaction, the neurologists may not speak to each other, but only to the Arbiter object or to the patient. Referring to FIG. 12a, an embodiment of a simplified example configuration 1201 of disease objects 1211 in a medical diagnostic and treatment advice system interacting with a patient 1260 is shown. Each disease object (e.g., headache specialist in one diagnosis) has within itself the methods of diagnosis that the disease authors feel are best for this disease in: e.g., Vertical Axis of Inquiry (VAI), Diagonal Axis of Inquiry (DAI) and Horizontal Axis of Inquiry (HAI). These methods include the best sequence to ask questions for each axis of inquiry, the disease time-line, and all other information necessary to diagnose that cause of headache.

The symptoms or patient health items inside a disease object are stored in a table that can be sorted by a number of different parameters. The different sort options contribute to many of the intra-disease objects diagnostic strategies.

Referring to FIG. 12b, one embodiment of an example configuration 1200 of a set of components used by the arbiter system and method is shown. In an example, an Arbiter object 1220 can function as a computerized neurologist who specializes in diagnosis of headaches. The Arbiter 1220 will be, in HAI and DAI modes, the one actually selecting which question to select from a set of disease objects 1210 which “vote” for a question they want to ask the patient 1260. In VAI mode, the questioning is “handed over” to one of the disease objects 1210. In certain embodiments, a medical diagnostic and treatment advice system 1205, such as described in U.S. Pat. No. 6,022,315, which is hereby incorporated by reference, communicates with the patient or proxy 1260 through an interface 1218, such as a graphical user interface, and with the arbiter object 1220. In certain embodiments, the medical diagnostic and treatment advice system 1205 includes a database 1215 that includes patient electronic medical records, the disease objects 1210 and a chief complaint object 1212 that can be considered as functioning as world class experts in each chief complaint. The medical diagnostic and treatment advice system 1205 is shown in FIG. 12b in two blocks for the sake of convenience. In certain embodiments, the medical diagnostic and treatment advice system 1205 is used to ask questions of the patient, including initial screening questions and problem screening questions, receive answers from the patient, establish patient health items (PHIs) based on the answers, and store the answers and PHIs in an electronic medical record for the particular patient. The chief complaint object 1212 identifies the main reason or complaints for seeking a consultation with the arbiter system. There are multiple ways of identifying a chief complaint, such as by anatomic system, alphabetically, and other ways such as described in U.S. Pat. No. 6,022,315, for example. Multiple expert diagnostic strategies 1230 are also in data communication with the Arbiter object 1220. The diagnostic strategies will be further described hereinbelow.

### The Patient

In this analogy, the Arbiter object 1220 functions as the entity that will control interaction with the patient 1260 while the system remains in HAI or DAI modes. The Arbiter may ask one question at a time or may provide the patient a list of questions in which the patient may “check off” the symptoms that apply. Each list is either static (the list does not change as the patient checks the symptoms that apply) or dynamic. In a dynamic list, every time the patient selects one symptom, the other symptoms may change.

### Patient Ombudsman

In this analogy, associated with the Arbiter 1220 is a Patient Ombudsman object 1250. The Patient Ombudsman object 1250 represents the patient 1260 and is always trying to reduce the number of questions that are asked of its client. This function is based on a proactive form of a Review of Systems (ROS) evaluation. The ROS questions are typically stem questions, e.g., the parent form of several different questions that can be evaluated as a group. In certain embodiments, the Patient Ombudsman object 1250 looks ‘backward’ in a hierarchical view of a patient health item and asks the most general or root PHI. The Patient Ombudsman object 1250 acts in the HAI, DAI and VAI modes.

For example, in the headache example, before the Arbiter 1220 asks for a particular eye sign or symptom, the Patient Ombudsman object 1250 will suggest a screening question to see if the patient has any eye or vision related complaints.

### Arbiter Method of Operation

The Arbiter object 1220 will start an evaluation session process by looking at a consultation. If this is the first time for the Arbiter to interact with the patient on this particular consultation, then the Arbiter will take a moment and initialize any data and setup any functionality it requires, before asking its first question. In certain embodiments, it is important to note that by the time the Arbiter system is invoked, the patient has already gone through usually three layers of screening questions designed to exclude very critical situations, a patient electronic medical record will have been opened or created, and all information available about this patient will have been passed or pushed (such as read from the electronic medical record from a prior consultation or session and placed into memory) to the Arbiter 1220 and the disease objects 1210, portions of which are described in U.S. Pat. No. 6,468,210. In certain embodiments, the chief complaint object 1212 is invoked after the initial screening questions are asked of the patient, to be sure that this is not a medical emergency, e.g., major trauma such as if there is a medical emergency or major trauma. The chief complaint object 1212 identifies the chief complaint based on information stored in the database 1215 and patient answers. In certain embodiments, after the chief complaint is identified several problem screening questions associated with the particular complaint are asked of the patient to screen out particular problems before continuing with the consultation. The chief complaint object 1212 then assemble an initial differential diagnosis which is provided to the Arbiter 1220.

Once that is completed, the Arbiter object will ask the patient the first question, based upon the data it already has in hand concerning this patient and this consultation. That is, the Arbiter starts the interaction by asking the disease objects to submit votes for the question or questions they would like to ask next. As a side note, the Arbiter can become more efficient over time and consultations with a patient, as the patient will build up a medical history such in a patient electronic medical record which the Arbiter can look at prior to starting the process. This will yield more efficient evaluations in some venues. For example, if a patient has had a prior appendectomy or tonsillectomy, then certain ailments can be pruned from consideration, as they do not apply—irrespective of what the presenting Chief Complaints or symptoms might initially indicate. Furthermore, answers to questions can be obtained from the patient electronic medical record rather than asking the patient, as applicable, to minimize redundant questioning, for example.

Once the patient has responded to the first question, the Arbiter object will drop into the Disease Qualifying section of operation which is part of the evaluation process for the Arbiter.

In certain embodiments, by default, the Arbiter will employ an Intent Modulation strategy, described hereinbelow, but can employ many other diagnostic strategies to evaluate the information provided during the consultation. Once the evaluation strategy has been selected, the Arbiter will then look to the disease objects and assess the “voting strength” of each one in order to pick the next question to ask. Part of this process involves segregating or separating some of the candidate diseases. Voting strength is varied based upon how successful each disease object has been in questioning the patient. That is, the more PHIs the patient has of a particular disease, the more voting strength that disease will acquire.

The Arbiter object is continually evaluating the session to determine if a change of strategy would be beneficial to the diagnostic process. If so, the Arbiter executes the most beneficial evaluation strategy and re-evaluates its lists of disease candidates as described previously. This operation will continue until the Arbiter has exhausted its list of strategies to employ, or an external event requires it to leave this mode of operation, such as Switching Axis of Inquiry or Diagnostic threshold has been reached, or other goal for the evaluation session has been reached.

The Arbiter system can be paused at anytime and the disease objects can be polled as to why they believe they are or are not the correct diagnosis.

### Intent Modulation

The purpose of Intent Modulation (IM) is a diagnostic strategy which is designed to eliminate the later stages of urgent diseases from the candidate list or, in the case of a patient who is in the later stages of an urgent disease process, to identify it as soon as possible and take the appropriate action(s).

Once the consultation has collected a sufficient amount of information, the Arbiter will invoke Intent modulation, which starts the qualification process with Late Stage Symptoms (LSS), of the disease(s) under consideration ranked by urgency, in certain embodiments. In certain embodiments, it should be noted that the Arbiter is by default in Horizontal Axis of Inquiry (HAI), unless otherwise noted.

### Description of the Arbiter Loop

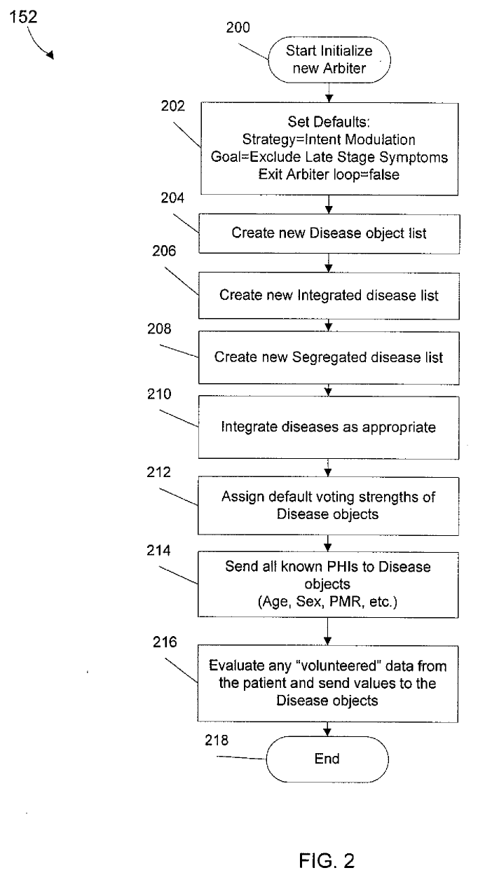

Referring to FIG. 1b, an embodiment of an Arbiter process 150 begins at a start state 151. Moving to an Initialize new Arbiter process 152, Arbiter process 150 initializes appropriate variables and prepares the new Arbiter process. Process 152 is further described in conjunction with FIG. 2.

Referring to FIG. 2, the Initialize new Arbiter process 152 begins at a start state 200 and moves to state 202 where the default strategy and mode variables are initialized. Specifically, in one embodiment, the strategy is set to Intent Modulation (IM), the mode is set to Exclude Late Stage Symptoms (LSS) and the Exit Arbiter Loop is set to False.

Next the initial disease objects are gathered into a list at a state 204. The disease objects that will make up the integrated list are assembled at state 206 and the disease objects that will make up the segregated disease list are assembled at state 208. In certain embodiments, all disease objects in the list (state 204) are initially integrated. By allowing only the integrated diseases to vote for the next question, the number of questions that the patient is asked is decreased. Note that the segregation/integration process is a dynamic one. As described below, if a segregated disease reaches or exceeds integration threshold, it will be integrated and then vote for what question to ask the patient in the next iteration of the Arbiter loop. Proceeding to state 210, process 152 now checks the segregated disease list to ensure that, based on the little that is known about this patient (e.g., patient electronic medical record, initial screening question answers, problem screening question answers), any diseases that should immediately be integrated are integrated. Once this is complete, state 212 assigns a default voting strength to each disease. In certain embodiments, the default voting strength of all disease objects is one. Other values are possible based upon, for example, the user's desire to exclude urgent or serious diseases to a high degree of certainty. In this case, those diseases that meet a threshold of urgency or seriousness would be given a higher initial voting strength.

Continuing at a state 214, the details that have been gathered at this stage of the consultation and any relevant data from the patient's electronic patient medical record (collectively, patient health items (PHIs)) are sent to the disease objects where the information is processed.

State 216 then checks to see if there is any additional information that the patient may have provided to the process 150. Arbiter initialization is concluded at an end state 218 and flow returns to the process 150 in FIG. 1b.

Referring back to FIG. 1b, state 154 is the start point of the actual Arbiter loop. This begin loop statement will execute the processes between it and state 164, the End Loop Statement, repeatedly so long as the variable Exit Arbiter Loop does not equal “true”.

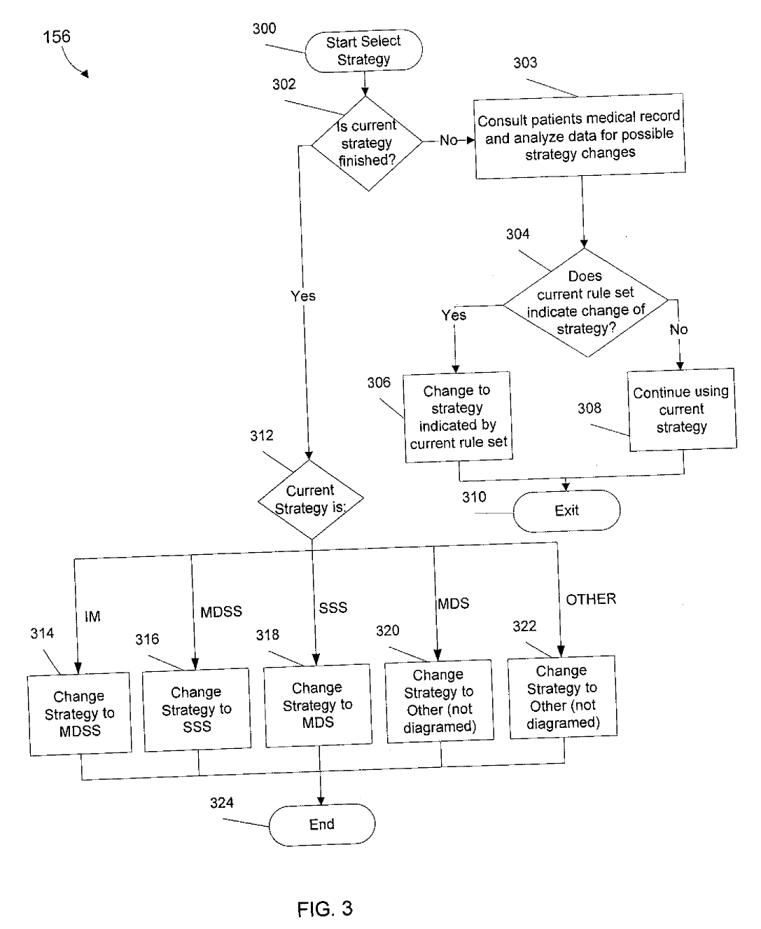

The first task in the loop is to select a strategy at a process 156. Process 156 is further described in conjunction with FIG. 3.

Referring to FIG. 3, the select strategy process 156 begins at start state 300 and moves to a decision state 302 to check if the current strategy is complete. If the current strategy is complete, process 156 proceeds to a decision state 312 to determine the current strategy and then change to a next strategy. If the current strategy is Intent Modulation (IM), process 156 proceeds to state 314 to change the current strategy to Mean Democratic Sine Strategy (MDSS). If the current strategy is MDSS, process 156 proceeds to state 316 to change the current strategy to Sequential Synergies Strategy (SSS). If the current strategy is SSS, process 156 proceeds to state 318 to change the current strategy to Mean Democratic Strategy (MDS). If the current strategy is MDS, process 156 proceeds to state 320 to change the current strategy to an Other strategy. If the current strategy is Other, process 156 proceeds to state 322 to change the current strategy to a further Other strategy. The Other strategies are reserved for future expansion of the Arbiter system. In other embodiments, the order of the strategies can be changed to a different order than that shown in FIG. 3. At the completion of any of the states 314, 316, 318, 320 or 322, process 156 ends at an end state 324.

Referring back to decision state 302 of FIG. 3, the select strategy process 156 checks if the current strategy is completed. In this example case, the process 150 has just begun with the default strategy of IM, so the result of the decision state would be “No”. Process 156 then proceeds to state 303 and checks the patient's medical record for information that may result is a strategy change. The patient's medical record is checked to determine if another strategy than the default diagnostic strategy should be used. An example of a situation for a change in diagnostic strategy could be, for example, if the patient was a reenter patient and the consultation had already progressed to a certain point. The next four paragraphs are further examples of what could initiate a change of diagnostic strategy.

In another example, in certain embodiments, the patient may have specified in their electronic medical record the degree of sensitivity or thoroughness that they preferred the system to function in. That is, some patients prefer a very “sensitive” workup in the sense that they want all possible diseases considered and do not mind spending several hours if necessary to diagnose their problem.

In another example, the patient's past use of the system may have reached a “meta” threshold for worry. For example, if the patient had too many problems all caused by infection, but in different organ system, the patient system, such as shown in U.S. Pat. No. 6,468,210 and U.S. Pat. No. 5,724,968, would make a “meta” recommendation and suggest that there may be a problem with the immune system. If the patient's HIV status is unknown, a recommendation for an HIV test could be made. If the patient were HIV+, a CD-4 count or viral load test would be suggested.

The patient may also, at any time, change the goal of the consultation in real time. For example, the patient may start a consultation with the intention of trying to arrive at a clinical diagnosis of their problem. If, for some reason, the patient needs to shorten the consultation, the goal can be changed for this consultation to exclude the urgent or serious causes of the problem and leave the diagnostic part for later.

If the patient has a medical record of diseases that may make the patient susceptible to other diseases, this information will be considered in the workup. From a Cause Disease Effect (CDE) view, the diseases which this patient's disease may cause have their prevalence increased in the diagnostic process. In addition, those diseases can be added to the differential even if, the patient's complaint(s) alone would not warrant that. For example, Diabetes predisposes the patient to a number of problems including atherosclerosis and kidney failure, and these will always be considered in the diagnostic session. Lupus makes the body's coagulation system much more likely to form clots. So diseases like acute coronary syndrome or pulmonary embolus must always be considered.

Advancing to a decision state 304 of FIG. 3, process 156 evaluates the current strategy and determines if a strategy change would be advised. Several examples of strategy changes are as follows.
1) If the patient decides that he/she does not want to continue toward diagnosis, but rather now exclude the worst case scenario or exclude the urgent or serious diseases, the strategy will be changed to exclude the urgent or serious diseases in the integrated area.
2) There is a symptom watch list on the user's screen. Frequently when using various time-based strategies, the next PHI which would normally be expected in a disease time-line will be placed in the Symptom or PHI watch portion of the screen. If, during the consultation, this PHI appears, the patient will click it and the strategy could be changed or the axis of inquiry could be changed, e.g., to diagnose in VAI. That is, both a change in Axis of Inquiry and strategy could occur at the same time.
3) There is also an area on the user's screen which asks the user to click if the patient's symptoms begin to get better. This is called the Nexus Point. Frequently in food poisoning or gastroenteritis, the patient knows immediately when he/she has “turned the corner”. If this is the case, the system may opt to stop the questioning and invoke a reenter consultation and ask the patient to reenter in say one hour.
4) Some strategies may specify that they are to be invoked for N questions, and if there are no positive responses, to switch to a new strategy.

Each disease object has a default sequence of questions for both VAI and HAI mode. In addition, each disease object contains the sequence of questions to be answered for different strategies including excluding the disease as quickly as possible. In the current example, the answer to the decision state 304 would also be “No”, and process 156 moves to state 308 and instructs the system to continue with the current strategy, Intent Modulation. Process 156 then moves to an exit function state 310 to end this task and returns to process 150 in FIG. 1b. However, in a different situation, if a strategy change is advised as determined at decision state 304, process 156 proceeds to state 306 and changes to the strategy indicated by the current rule set and then moves to the exit function state 310.

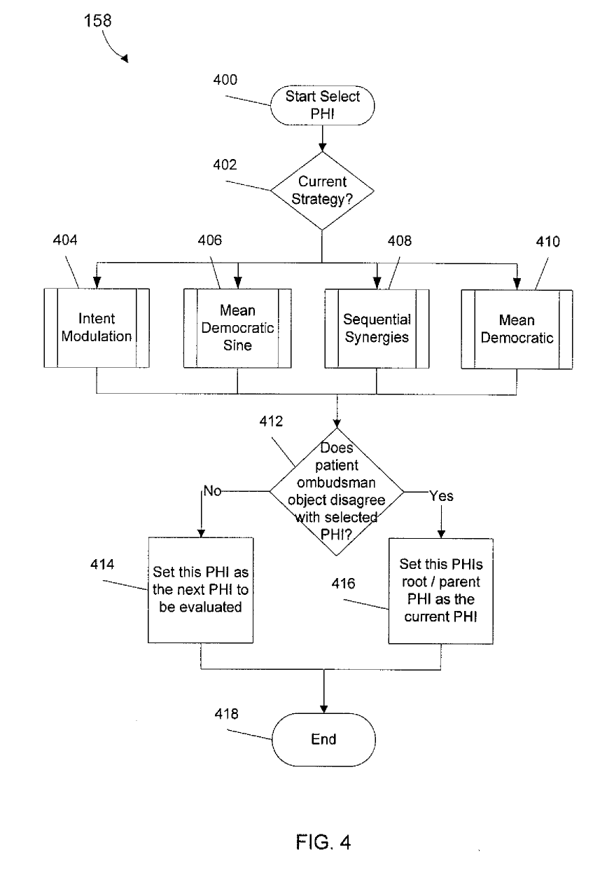

On FIG. 1b, process 158 describes a method of selecting the next Patient Health Item (PHI) that will be presented to the user. This process is further described in conjunction with FIG. 4.

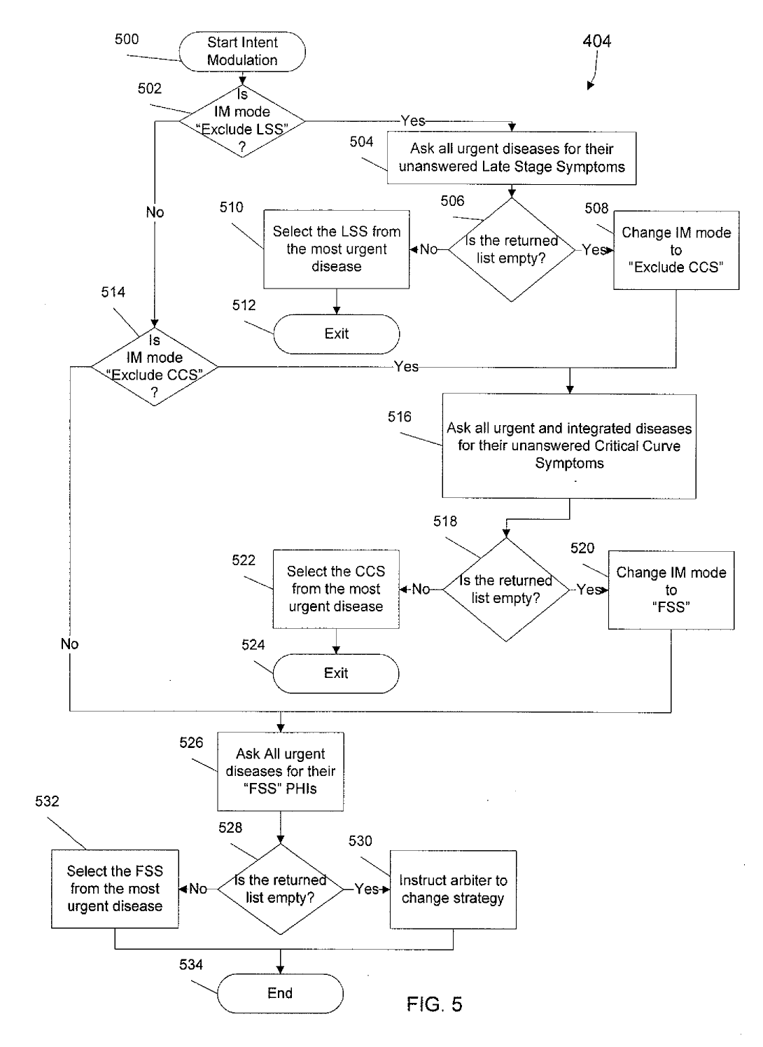

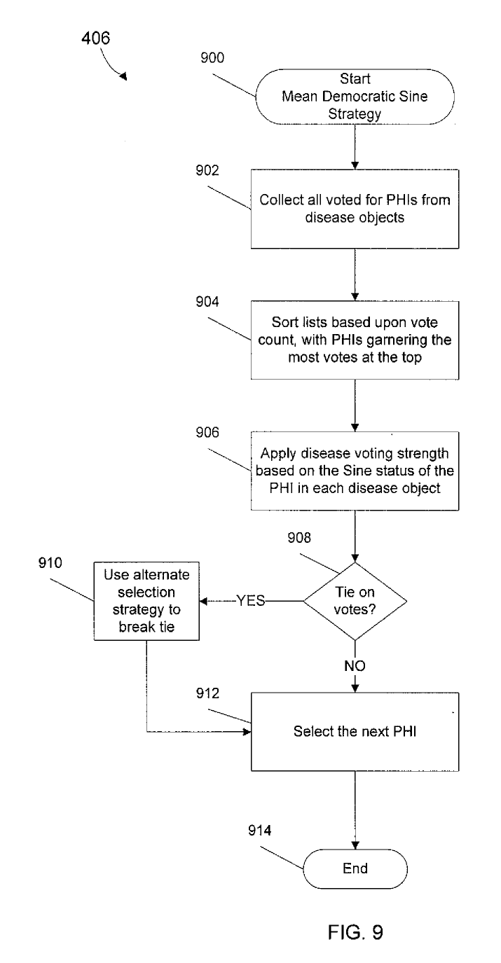

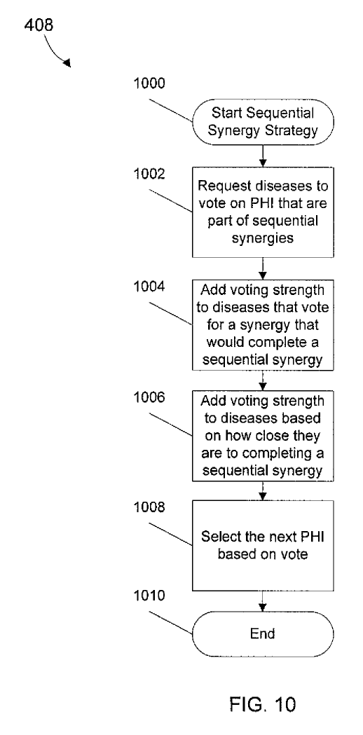

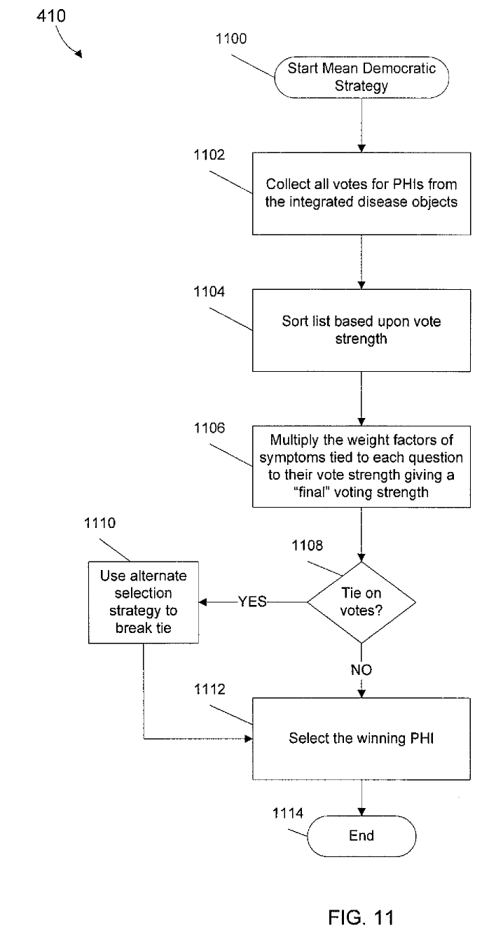

Referring to FIG. 4, the process 158 for selecting the next PHI will be described. Process 158 begins at a start state 400 and proceeds to a decision state 402 where the current strategy affects the process by branching to a specific routine for each possible strategy. Note that FIG. 4 does not reflect a complete list of possible strategies but rather a subset that is adequate for this example. If the current strategy is IM, an IM process 404 is executed, which is further described in conjunction with FIG. 5. If the current strategy is DMSS, a DMSS process 406 is executed, which is further described in conjunction with FIG. 9. If the current strategy is SSS, a SSS process 408 is executed, which is further described in conjunction with FIG. 10. If the current strategy is MDS, a MDS process 410 is executed, which is further described in conjunction with FIG. 11. At the conclusion of performing any of the processes 404, 406, 408 or 410, process 158 continues at a decision state 412 to determine if the Patient Ombudsman objects to the selected PHI. If not, process 158 advances to state 414 and sets the selected PHI as the next PHI to be evaluated. If the Patient Ombudsman object disagrees with the selected PHI as determined at the decision state 412, process 158 moves to state 416 and sets the selected PHI's root or parent PHI as the current PHI. At the completion of either state 414 or 416, process 158 completes at an end state 418.

Referring to FIG. 5, the Intent Modulation process 404 will now be described. The discussion below utilizes concepts of disease timelines. Various characteristics, such as aspects of symptoms or patient health items, of a particular disease can be plotted or otherwise associated with time, such as described in U.S. Pat. No. 6,569,093, which is hereby incorporated by reference in its entirety. Several categories of PHIs have been established for disease timelines: late stage symptoms or PHIs, critical curve PHIs, first PHI, and first significant symptom or PHI which are considered to be absolute for a particular disease. The critical curve PHIs are the PHIs that occur second to last in a particular disease timeline. these typically occur when the disease process is accelerating and can indicate the need for very rapid intervention. A next expected PHI is a relative term for a disease timeline.

Beginning at a start state 500 of FIG. 5, process 404 advances to a decision state 502 where a check is made to see if the current mode is “Exclude LSS”. In an example use of process 150, Exclude LSS is the mode that was set back on FIG. 2 at state 202, and if it has been not changed, the answer would be “Yes” causing process 404 to advance to state 504 and request that the urgent disease objects provide a list of their unanswered Late Stage Symptoms (LSS). These are the PHIs that would be expected in the later stages of the diseases. Process 404 then proceeds to a decision state 506 to determine if the returned list is empty or if it contains PHIs to process. If the list is not empty, as determined at the decision state 506, process 404 proceeds to state 510 and selects the LSS from the most urgent disease. This process 404 is now completed by an exit state 512. However, if the process had already asked all of the LSS PHIs, the returned list would be empty, as determined at the decision state 506, and process 404 would advance to state 508. At state 508, process 404 changes the mode to “Exclude Critical Curve Synergies (CCS)” in order to begin excluding Critical Curve Synergies.

Returning to decision state 502 of FIG. 5, if the IM mode is not Exclude LSS, process 404 proceeds to a decision state 514 to determine if the IM mode is “Exclude CCS”. If the IM mode is Exclude CCS or the mode was changed to Exclude CCS at state 508, process 404 advances to state 516. At state 516, process 404 requests that the urgent and integrated disease objects provide a list of their unanswered CSS. Process 404 then proceeds to a decision state 518 to determine if the returned list is empty or if it contains PHIs to process. If the list is not empty, as determined at the decision state 518, process 404 proceeds to state 522 and selects the CCS from the most urgent disease. This process 404 is now completed by an exit state 524. However, if the process had already asked all of the CCS PHIs, the returned list would be empty, as determined at the decision state 518, and process 404 would advance to state 520. At state 520, process 404 changes the IM mode to “First Significant Symptom (FSS)” in order to begin processing First Significant Symptoms.

Returning to decision state 514 of FIG. 5, if the IM mode is not Exclude CCS or if the IM mode was changed to FSS at state 520, process 404 proceeds to state 526, At state 526, process 404 asks all of the urgent diseases for their FSS PHIs. Proceeding to a decision state 528, process 404 checks whether the list is empty. If the list is not empty, process 404 continues at state 532 and chooses the FSS PHI from the most urgent disease. If the list is empty, as determined at decision state 528, process 404 advances to state 530 and instructs the Arbiter to change strategy. At the completion of either state 530 or 532, process completes at an end state 534.

Voting strength is not shown explicitly in FIG. 5. In Intent Modulation, those diseases that are urgent have their initial voting strength increased, such as described in conjunction with state 212 (FIG. 2) above. Each time a PHI is established in a disease, its voting strength is increased. Each time a PHI is established that does not occur in a disease object, its voting strength may be decreased. There are other criteria that can increase or decrease voting strength.

Referring to FIG. 9, the Mean Democratic Sine Strategy (MDSS) process 406 will now be described. Beginning at a start state 900, process 406 advances to state 902 where the process 406 collects all the voted for PHIs from the Disease Objects (DOs). Proceeding to state 904, process 406 sorts the list of voted for PHIs placing the ones with the most votes first, in certain embodiments. Next at state 906, process 406 applies the disease voting strengths for each disease based on the sine status of the PHIs. Each PHI in a disease object is given a “sine” status. “Sine” is taken from “Sine qua non” or without which there is nothing. This is in addition to the diagnostic weight to be added or subtracted from the disease score if the PHI is present or not present. Pathonemonic is the highest “sine” level, which means if the patient has this PHI, they have the disease. For example, a patient who sees fortification figures (top of a turret) before or with a headache always has a migraine headache. “Sine qua non major” means that a clinician would not generally diagnose a disease if this PHI or symptom is not present. A good example is Acute Coronary Syndrome (ACS), where generally, without chest pain the clinician would not consider that diagnosis. Even this has to be qualified because it is possible to have ACS without pain in an elderly patient especially with diabetes. “Sine qua non minor” means that a clinician would generally expect this PHI to be present to diagnose the disease. In certain embodiments, a disease object may specify that 2 out of 3 sine qua non majors must be present for the diagnosis to be made. This is somewhat analogous to the Jones Criteria to diagnose rheumatic fever.

Advancing to a decision state 908 of FIG. 9, process 406 checks to determine if a tie has occurred in the PHI votes. If a tie has occurred, an alternate strategy is called to break the tie at state 910. At the completion of state 9 10 or if there is no tie in the votes, the winning PHI is selected at state 912 and the process 406 completes at an end state 914.

Referring to FIG. 10, the Sequential Synergy Strategy (SSS) process 408 will now be described. In the sequential synergy strategy, the Arbiter asks the disease object if any of the PHIs that have already been established in the patient are involved in one or more sequential synergies. If the answer is “yes”, then the Arbiter asks the disease object to ask a question that would establish a sequential synergy. For a sequential synergy, there are some diseases in which the onset and progression of symptoms is very important in diagnosis. For example, acute appendicitis has a well know sequence of symptoms that are important to diagnose it. They are generally as follows: first the patient may become anorexic (not want to eat) or may develop a very non-localized upper abdominal pain. This is followed by nausea and perhaps vomiting, and then the pain moves to the right lower quadrant (RLQ) of the abdomen.

In certain embodiments, the Arbiter requests the first sequential synergy in a disease timeline, after the urgent diseases have been excluded. If the patient does have the other PHI involved in the sequential synergy, then the voting strength is increased. Beginning at a start state 1000, process 408 advances to state 1002 and requests that the disease objects vote only for PHIs that are part of sequential synergies. Advancing to state 1004, process 408 then adds voting strength to diseases who have voted for a PHI that completes a sequential synergy. Moving to state 1006, process 408 adds voting strength to diseases that have voted for PHIs that are near to completing a synergy. Proceeding to state 1008, process 408 selects the elected PHI based on the vote and then completes at an end state 1010.

Referring to FIG. 11, the Mean Democratic Strategy (MDS) process 410 will now be described. Beginning at a start state 1100, process 410 advances to state 1102 to collect votes for PHIs from diseases in the integrated disease list. Advancing to state 1104, process 410 sorts the PHIs based upon the voting strength of the disease(s) voting for each PHI. Continuing at a state 1106, process 410 multiplies the weight factors of each PHI to the vote strength to determine the final votes for each PHI. Proceeding to a decision state 1 108, process 410 checks for the possibility of a tie, and uses state 1110 to break that tie by an alternative strategy if necessary. At the completion of state 1110 or if there is no tie in the votes, the winning PHI is selected at state 11 12 and the process 410 completes at an end state 1114.

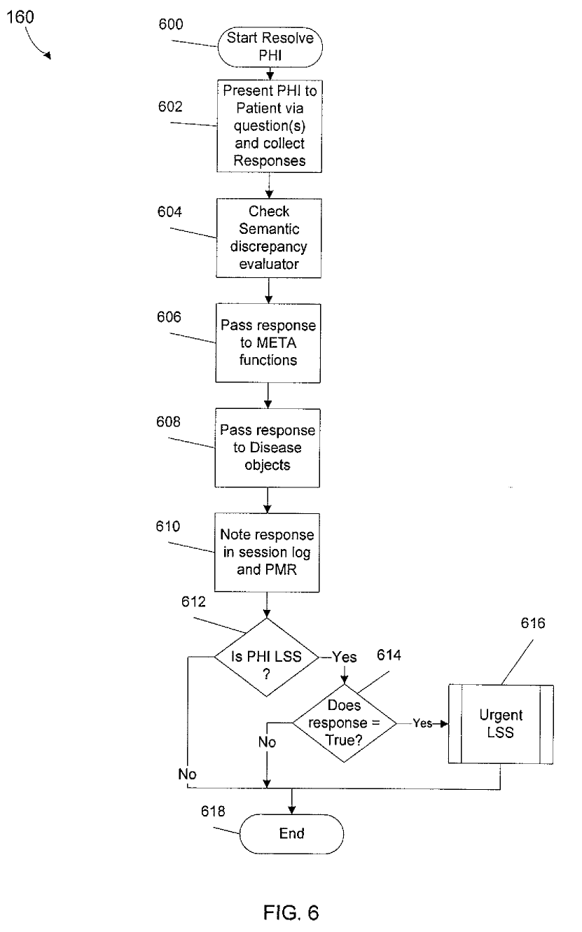

Referring back to FIG. 1b, at the completion of process 158, a resolve PHI process 160 begins the process of resolving this new PHI. This is further described in conjunction with FIG. 6.

Referring to FIG. 6, the process 160 for resolving the selected PHI will be described. Process 160 begins at a start state 600 and proceeds to a state 602 to present the PHI to the user or patient and collect a response. The details of how this is accomplished changes depending on the current embodiment; however, the results are handled in the same way. Advancing to state 604, process 160 checks a semantic discrepancy evaluator to ensure that this result does not contradict information already known by the system. This helps the system to identify patients that may have an altered or an abnormal level of consciousness. For this example an assumption is made that the test is passed. Proceeding to state 606, the indicated response is passed to a set of Meta analysis functions that are running as independent threads alongside the main process. In addition to the cause Meta mentioned above regarding too many instances of problems caused by infection, there are several other Meta functions discussed in Applicant's other patents, such as U.S. Pat. No. 5,724,968, which is hereby incorporated by reference. The very fact that a patient is consulting the system for a complaint such as headache too many times per unit of time, would make the system make a “meta” recommendation that perhaps a CT or MRI of the brain should be obtained to rule out a brain tumor.

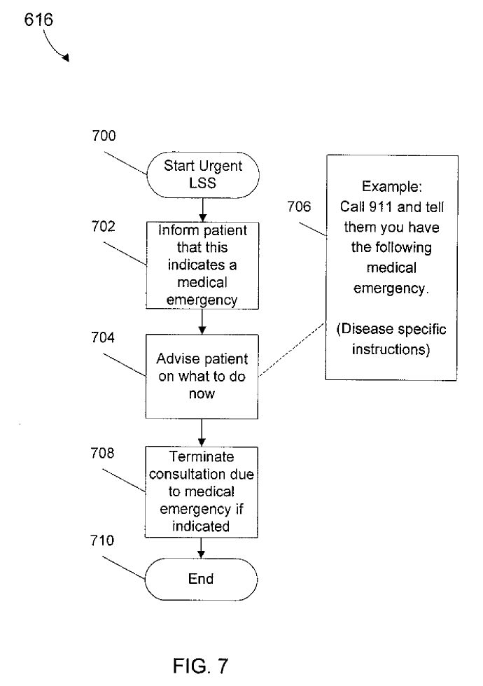

Continuing at state 608 of FIG. 6, the response is passed to the disease objects where each disease that has this PHI will score the result and calculate its new weight and probabilities. Moving to state 610, the question that was posed to the user and the response collected is stored into the patient's patient medical record (PMR) or electronic medical record (EMR) along with a time-date stamp of the interaction. Advancing to a decision state 612, process 160 then checks to see if this PHI is a LSS of an urgent disease. If true, then process 160 moves to a decision state 614 to determine if the result of that LSS indicated that a Late Stage PHI in an urgent disease is actually present. If true, then process 160 advances to an Urgent LSS process 616. Process 616 is further described in conjunction with FIG. 7 below. If the result of either decision state 612 or decision state 614 is not true or at the completion of the Urgent LSS process 616, process 160 completes at an end state 618.

Referring to FIG. 7, the Urgent LSS process 616 will now be described. Beginning at a start state 700, process 616 advances to state 702 where the patient is informed of the presence and the meaning of the urgent LSS information (e.g., a medical emergency exists). Proceeding to state 704, process 616 provides the patient with information on what to do next. In certain embodiments, as indicated at exemplary state 706, this recommendation can range from general medical advice, or advising the patient to consult their physician, to instructions to immediately activate the local 911 emergency system. Advancing to state 708, process 616 stops the consultation if a medical emergency so indicates. Process 616 then completes at an end state 710 and returns to the end state 618 shown in FIG. 6.

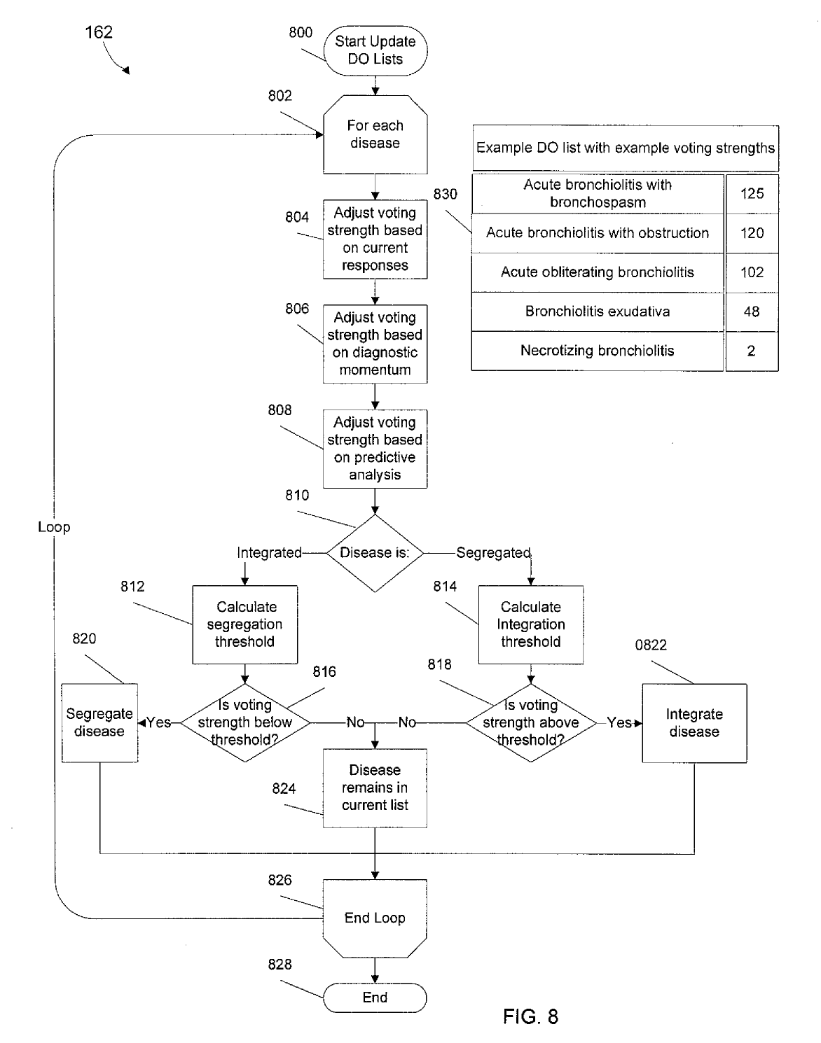

Referring back to FIG. 1b, at the completion of process 160, an update disease objects lists process 162 is invoked. This is further described in conjunction with FIG. 8.

Referring to FIG. 8, the process 162 for updating the disease object lists will be described. Process 162 begins at a start state 800 and proceeds into a loop starting at state 802. This loop executes for each disease, ending at a state 826 when the last disease has been processed. Moving to state 804, process 162 adjusts the voting strength of the current disease based on the current response. State 806 then adjusts the voting strength of this disease based on its diagnostic momentum. State 808 then executes a predictive analysis or “what if” session similar to a computerized chess algorithm to see what might happen, and the diseases voting strength is then modified based on this prediction. Predictive analysis refers to the system's ability to, e.g., do a what if analysis of a disease object. In certain embodiments, see that the disease object needs only a few PHIs to be diagnosed (increase of voting strength) or that, e.g., after a certain number of questions, only a few PHIs of that disease are present (decrease voting strength). Another example would be if all of the PHIs of a disease object were to be present, would it be possible to reach diagnosis, if not then the voting strength could be zero, in this example, under certain conditions.

Proceeding to a decision state 810, process 162 checks to determine if the current disease is integrated or segregated. If the disease is segregated, process 162 advances to state 814 where the integration threshold is calculated. There are several parameters that affect the segregation and integration of diseases. First, the score of a disease expressed as a percentage determines whether the disease is integrated so as to allow it to ask questions. A disease is integrated when the diagnostic score reaches or exceeds a threshold, typically a percentage of the score required to have a clinical diagnosis. Note that this threshold depends on the sensitivity factor set. If sensitivity is set high, more diseases are integrated. In addition to the score itself, the first derivative of the score, that is, the rate at which the score is increasing is used to integrate diseases, just as it is used to switch the Axis of Inquiry. In addition, the disease object contains within itself a table that lists the combination of PHIs that should integrate the disease. This is typically some number of sine qua non PHIs or combinations of sine qua non major and minor PHIs. In addition, the voting strength of diseases is also used to establish the integration threshold. The voting strength of a disease again reflects how much “attention” the Arbiter gives that disease object. Typically, if the patient is answering all of the questions in the affirmative, that is, the patient has all of the symptoms of the disease, then the voting strength is increased. This again tends to reduce the number of questions that patient has to answer. Thus the decision to integrate a disease object can be based on the score, the diagnostic momentum, or the combination of sine qua non major or minor PHIs specified by the author.

Continuing at a decision state 818 of FIG. 8, process 162 checks the threshold against the current disease to determine if the voting strength of the current disease has gone up enough to reach or exceed the integration threshold. If so, process 162 proceeds to state 822 to move this disease from the segregated list into the integrated list. Being in the integrated list allows the disease to submit votes on subsequent PHIs. If the voting strength is not above the threshold, as determined at decision state 818, process 162 advances to state 824 where the current disease remains in the current list. In one embodiment, if the disease object determines, during a self-evaluation, that even if all its remaining PHIs were to be established and associated synergies applied to obtain the maximal diagnostic score that a diagnostic threshold could not be reached or exceeded, then the disease is excluded from further diagnostic consideration.

Returning to decision state 810 of FIG. 8, if the current disease is integrated, process 162 advances to state 812 where the segregation threshold is determined. In certain embodiments, the segregation threshold is the same value as the integration threshold, and is indicative that the current disease is presently integrated but the score and/or voting strength is to be compared against the threshold to determine if it should moved to the segregated list. Continuing at a decision state 816, process 162 checks the threshold against the current disease and to determine if the voting strength of the current disease has gone down enough to be below the segregation threshold. If so, process 162 proceeds to state 820 to move this disease from the integrated list into the segregated list. Being in the segregated list does not allow the disease to submit votes on subsequent PHIs. If the voting strength is not below the threshold, as determined at decision state 816, process 162 advances to state 824 where the current disease remains in the current list. As described above, in one embodiment, if the disease object determines that even if all its remaining PHIs were to be established and associated synergies applied to obtain the maximal diagnostic score that a diagnostic threshold could not be reached or exceeded, then the disease is excluded from further diagnostic consideration. At the completion of either state 822, state 824 or state 820, process 162 advances to the end of the loop at state 826 and then returns execution to the top of the loop at state 802 where the loop repeats for the next disease. Once the disease object list is completed at end loop state 826, process 162 ends at an end state 828. An example disease object (DO) list with example voting strengths is shown at blocks 830.

Referring back to FIG. 1b, at the completion of process 162, process 150 reaches the end of loop statement at state 164. If the End Arbiter Loop is not “true”, the Arbiter loop repeats starting again at state 154. This Arbiter loop continues until an interrupt occurs that causes the loop to stop. There are many conditions or goals that can cause the loop to stop. A few examples of the conditions are as follows:
1. The user can end the session and thereby cause the program to terminate.
2. A medical emergency can terminate the session, thus causing the program to close down.
3. A diagnosis can be made that would end the consultation.

In any case, once the loop completes at state 164, process 150 advances to state 166 and takes an appropriate action based on the cause and condition of the system upon completion of the Arbiter loop. Examples of the appropriate action could be calling an emergency telephone number (e.g., 911), scheduling a reenter consultation, stopping to perform a self or assisted physical examination maneuver, or stopping to perform a home lab test. Process 150 concludes at an end state 168, and control then passes back to the main diagnostic system.

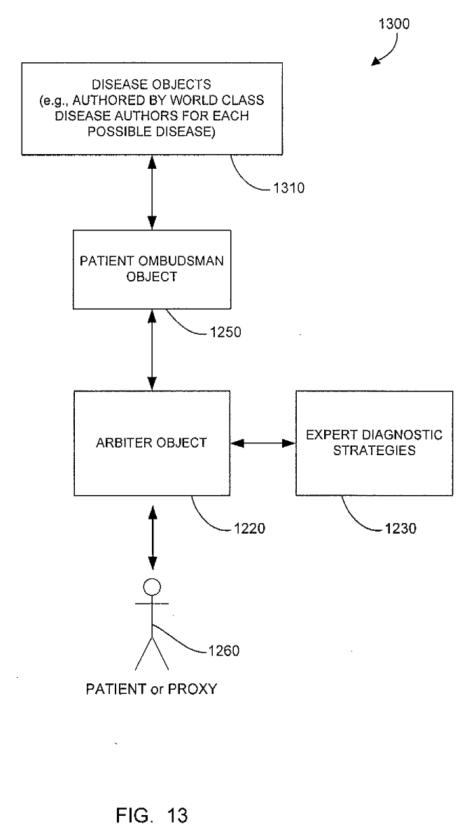

FIGS. 13, 14 and 15 illustrate concepts of each of the three axes of inquiry, respectively. Certain components of the medical diagnostic and treatment advice system 1205 (FIG. 12b) are not shown for the sake of conciseness. Referring to FIG. 13, one embodiment of an example configuration 1300 of a set of components used by the arbiter process operating in the horizontal axis of inquiry (HAI) mode is shown. In HAI mode, all disease objects 1310 (each a specialist in a particular disease) “vote” for the questions that they want to ask of the patient 1260. The HAI mode can be considered to be a high level mode of inquiry. The Arbiter 1220 decides which question is the next one to be asked of the patient. In certain embodiments, the Arbiter 1220 is not democratic in approach, but looks first at the late stage of an urgent disease, then attempts to exclude serious diseases, and then favors most common diseases over least common diseases. In other embodiments, other evaluation strategies, such as described above, can be utilized. The Patient Ombudsman object 1250 can communicate with the Arbiter 1220 to disagree with a selected PHI or to suggest a particular PHI. When the Patient Ombudsman object 1250 sees that many of the disease objects 1310 in diagnostic consideration would be voting for rather specific symptoms each of which is dependent upon a more general symptom, the Patient Ombudsman object 1250 would suggest that more general question to the Arbiter.

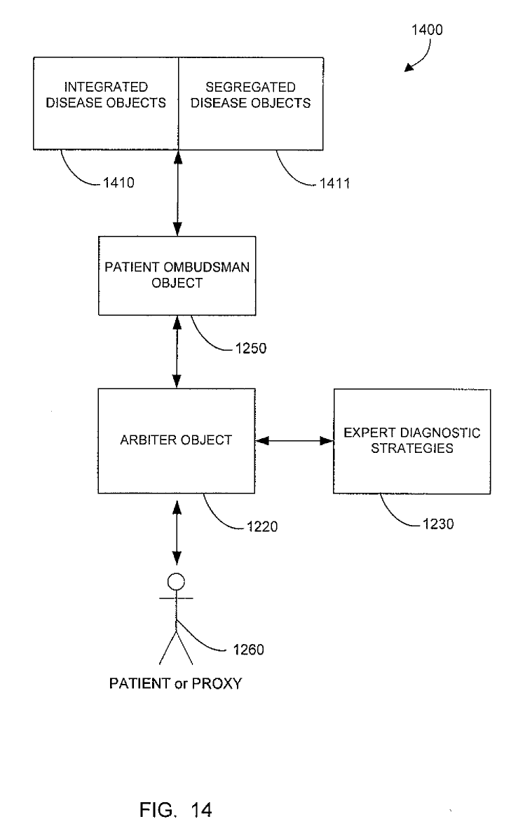

Referring to FIG. 14, one embodiment of an example configuration 1400 of a set of components used by the arbiter process operating in the diagonal axis of inquiry (DAI) mode is shown. In DAI mode, some disease objects (specialists) are segregated based on diagnostic likelihood. Separating the disease objects into an integrated group 1410 and a segregated group 1411 of disease objects provides the ability to restrict the voting of the objects in the segregated group 1411. The DAI mode can be considered to be a middle level mode of inquiry. All disease objects “listen” to or monitor a question asked of the patient 1260 and a response to the question, and score their diseases accordingly. Disease objects move in and out of the segregated group dynamically as questions are answered. Use of the DAI mode decreases the number of questions asked of the patient. The Patient Ombudsman object 1250 can communicate with the Arbiter 1220 to disagree with a selected PHI or to suggest a particular PHI.

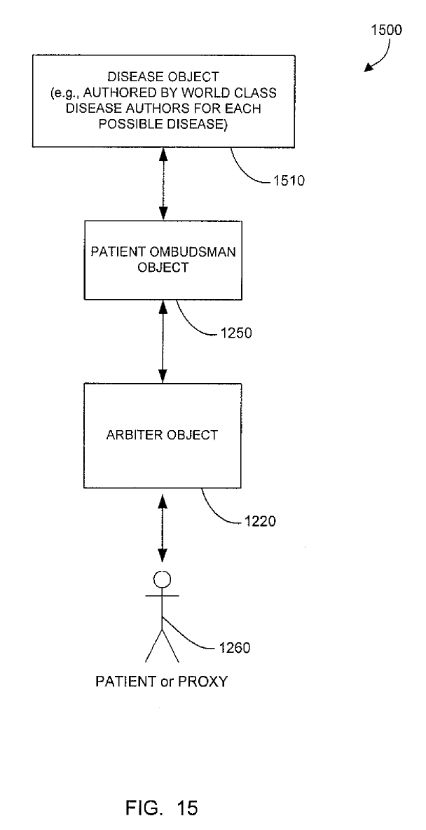

Referring to FIG. 15, one embodiment of an example configuration 1500 of a set of components used by the arbiter process operating in the vertical axis of inquiry (VAI) mode is shown. In VAI mode, one disease object 1510 believes that its corresponding disease is the most likely diagnosis and notifies the Arbiter 1220 accordingly. The Arbiter then allows this disease object to ask the questions preferred by the author of the disease object of the patient. The VAI mode can be considered to be a low level mode of inquiry. The Patient Ombudsman object 1250 communicates with the Arbiter 1220 to disagree with a selected PHI or to suggest a particular PHI. As in the DAI mode, all other disease objects “listen” to or monitor a question asked of the patient and a response to the question, and score their diseases accordingly. In certain embodiments, questioning proceeds in VAI mode until a diagnosis is reached or criteria are met such that the current disease object no longer qualifies to be only object questioning the patient. For example, another disease object could be selected to be in VAI mode in place of the current disease object, or the mode could be changed to DAI mode.

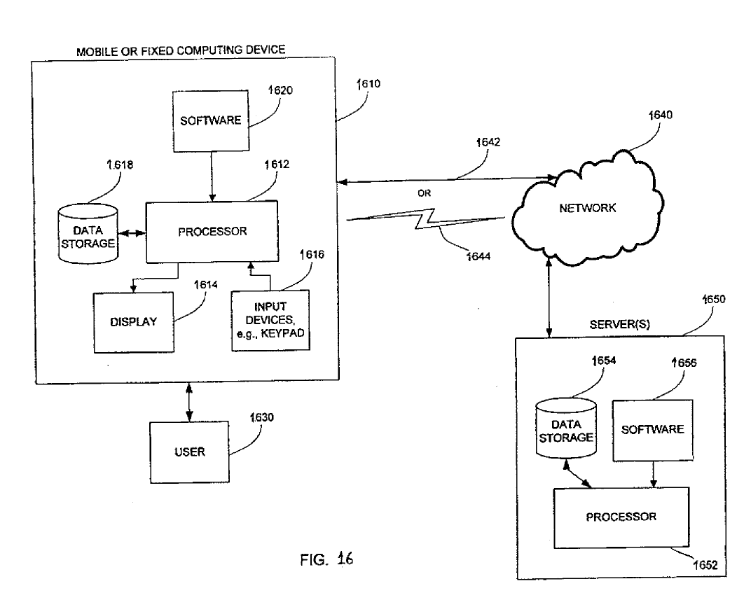

Referring to FIG. 16, an exemplary configuration 1600 of components of another embodiment of the MDATA system will now be described. A mobile or fixed computing device 1610 is operated by a user 1630. The computing device 1610 can be a handheld computing device or other portable computing device such as a Palm, Pocket personal computer (PC), Linux based handheld, PDA, Tablet PC, or PC having a display. The computing device 1610 in certain embodiments operates in a stand-alone (independent) manner. In other embodiments, the computing device 1610 is in communication with one or more servers 1650 via a network 1640. The server(s) include one or processors 1652, data storage 1654 and system software 1656 executed by the processor(s). In certain embodiments, the data storage 1654 stores one or more databases used by the system, and stores patient medical records. The processor(s) 1652 are in communication with the database(s) via a database interface, such as structured query language (SQL) or open database connectivity (ODBC). In certain embodiments, the data storage 1654 is not included in server(s) 1650, but is in data communication with the server(s) via the database interface. The connection from the computing device 1610 to the network 1640 can be a wireless or a satellite connection 1644 or a wired or direct connection 1642. In certain embodiments, the server(s) are part of a web site, such as on an intranet or the Internet.

When the computing device 1610 is connected with the server(s) 1650, the web site may optionally provide updates on new disease information or about new laboratory tests, special studies, imaging modality of choice and treatment of choice. In addition, the computing device 1610 can optionally be linked to the network 1640 to allow instantaneous reporting of and downloading information about, for example, possible epidemics or the use of weapons of mass destruction (WMD). In another embodiment, the computing device 1610 runs only when connected to the server(s) 1650.

The computing device 1610 includes a processor 1612, a display 1614, and one or more input devices 1616. The processor 1612 is in data communication with a data storage 1618 for storing one or more databases having medical data used by the system. In certain embodiments, the data storage 1618 stores patient medical records, such as a patient electronic medical record. System software 1620 is executed by the processor 1612. The system software 1620 can include an application graphical user interface (GUI). The application GUI can include a database interface to the data storage 1618 of the computing device. In certain embodiments, the software is loaded from the data storage 1618. In embodiments where the computing device 1610 communicates with a web site, the processor utilizes browser software in place of or in addition to the software 1620.

### Object-Based Diagnosis

In computer software terms, an object is combination of data and processes that manipulate the data. The data are said to be “encapsulated,” meaning that the data is hidden, so that a user of the object only sees processes that can be invoked. Using an object's processes, one can then manipulate the data without having to know the exact location and format of the data. When more than one copy of the object is required, one can make copies of the data, but use the same process set to manipulate each of the copies as needed. This set of processes can then be thought of as an “engine” that controls or represents the objects' behavior, whether there are 10 or 10,000 object copies, for example.

This section describes a diagnostic paradigm that uses software objects to establish a broad, generalized software environment for medical diagnosis, which is used to define and develop the programming elements of medical diagnosis. The objects are then used to guide and control the diagnostic process, to conduct the patient interview, to perform related analytical tasks, and to generate diagnoses. A software object is a fundamental software structure that can be used to organize the processes and data of a computer program in such a way as to make possible very complicated applications. This description will discuss novel uses of object oriented programming (OOP) in medical diagnosis, such as the use of software objects for the purpose of fully automated medical diagnosis, the entire/overall method of dynamically assembling the components of diagnosis in the form of objects, and then letting the objects interact to compute a result such as a diagnosis.

Defining and creating software objects is well-known to any programmer trained in object-oriented programming. Using an OOP-capable compiler, the programmer defines the data that represent the object and the actions that the object can perform. At run time, the program creates an object, supplies the data that define the object, and then manipulates the object using the object actions. The program can create any number of objects as needed. Each object can be independently initialized, manipulated, and destroyed.

In an object-based (OB) method, software objects are used as active, intelligent agents that represent all of the functionality and all of the data in suitably organized roles. It is important to note in this metaphor that all of the disease objects, which are virtual “specialists” for a single disease, are allowed to monitor the questions and answers of other objects. Each object may get a turn at evaluating the patient data in terms of its own symptom pattern.

As an actual patient symptom set is built up, disease objects judge themselves and return a probability that they are the correct diagnosis. The emergent effect is a patient interview and a diagnostic evaluation that, by design, constantly stays focused on the most likely set of diseases of the patient. Carefully focused questions are used to eliminate or reduce the likelihood of diseases, to promote others into the “realm of suspicion,” and to expand the search in a promising direction, based on the data being obtained from the patient.

A software “object” is basically a data structure plus associated processes that can do things with, for or to the data. An important property of an object is that the object's data can be hidden behind the object's processes, so that the outside user of the object can only see and use object processes that can be invoked to access the data. The object is said to “hide” data; it provides the powerful ability of decoupling the world that uses an object from the object itself.

### CONCLUSION

While specific blocks, sections, devices, functions and modules may have been set forth above, a skilled technologist will realize that there are many ways to partition the system, and that there are many parts, components, modules or functions that may be substituted for those listed above.

While the above detailed description has shown, described, and pointed out the fundamental novel features of the invention as applied to various embodiments, it will be understood that various omissions and substitutions and changes in the form and details of the system illustrated may be made by those skilled in the art, without departing from the intent of the invention.

What is claimed is:

1. A computerized arbiter method utilized during an evaluation session in a medical diagnostic system having a computing device, the method comprising:
asking general questions associated with a list of candidate diseases of a patient using a high level mode of inquiry by use of a user interface associated with a computing device;
selecting a set of most likely diseases based on the responses to the general questions;
asking questions focused on the set of most likely diseases using a middle level mode of inquiry;
selecting a most likely disease based on the responses to the questions from the middle level of inquiry; and
asking questions focused on the most likely disease using a low level mode of inquiry,
wherein a sequence of questions corresponds to one of a plurality of evaluation strategies, wherein the set of most likely diseases is divided into one class that is allowed to vote for the next best question which is to be asked of the patient or into another class that is not allowed to vote for the next best question, wherein the diseases that are in the class that cannot vote for the next best question add a weight to a disease score, the weight corresponding to a response for a question asked by another disease, and wherein questions are asked until a goal of the evaluation session has been reached.

2. The method of claim 1, wherein one of the evaluation strategies is intent modulation which eliminates the later stages of urgent diseases from the list of candidate diseases.

3. The method of claim 1, wherein the responses to the questions are stored in a patient electronic medical record and are used to establish patient health items (PHIs), and wherein each candidate disease is associated with one or more PHIs, and each PHI is associated with one or more questions.

4. The method of claim 3, wherein one of the evaluation strategies is mean democratic sine which determines the next best question by a voting process wherein the sine status of a PHI and disease pair is factored into the voting strength of the diseases.

5. The method of claim 3, wherein one of the evaluation strategies is a sequential synergy strategy which gives more voting strength of priority to those PHIs that complete or nearly complete a sequential synergy.

6. The method of claim 1, wherein the diseases that are in the class that are allowed to vote for the next best question add a weight to a disease score.

7. The method of claim 1, wherein the next best question is the question that advances the evaluation session to reach a correct diagnosis at the earliest point in time with the fewest number of questions.

8. The method of claim 1, wherein the number of questions asked of the patient is reduced based on use of the middle level of inquiry where the class that is not allowed to vote for the next best question does not contribute potential questions to be asked of the patient.

9. The method of claim 3, wherein questions corresponding to late stage PHIs of urgent diseases are asked first so as to diagnose or exclude those diseases that have a limited therapeutic window of opportunity.

10. The method of claim 1, wherein a particular one of the plurality of evaluation strategies can be changed responsive to a clinical situation of the patient.

11. The method of claim 3, wherein a particular one of the plurality of evaluation strategies is selected depending upon a past medical history of the patient as stored in the patient electronic medical record.

12. The method of claim 1, wherein a particular one of the plurality of evaluation strategies is selected depending upon the patient's previous responses in a consultation.

13. The method of claim 1, additionally comprising selecting from the questions voted on by the diseases in diagnostic consideration.

14. The method of claim 1, wherein the method does not permit diseases of the class not allowed to vote of the set of most likely diseases to suggest questions to ask of the patient during the evaluation session.

15. A computerized medical diagnostic system, the system comprising:
a computer storage storing a list of candidate diseases, each candidate disease associated with one or more questions;
a computing device in data communication with the computer storage, the computing device performing software instructions to:
ask general questions associated with the candidate diseases of a patient using a high level mode of inquiry;
select a set of most likely diseases based on the responses to the general questions;
ask questions focused on the set of most likely diseases using a middle level mode of inquiry;
select a most likely disease based on the responses to the questions from the middle level of inquiry; and
ask questions focused on the most likely disease using a low level mode of inquiry,
wherein the set of most likely diseases is separated into a first class that is allowed to vote for the next best question which is to be asked of the patient or into a second class that is not allowed to vote for the next best question, wherein the diseases that are in the second class add a weight to a disease score, the weight corresponding to a response for a question asked by another disease, and wherein questions are asked until a goal of the evaluation session has been reached.

16. The system of claim 15, wherein the responses to the questions are stored in a patient electronic medical record and are used to establish patient health items (PHIs).

17. The system of claim 16, wherein the diseases in the list of candidate diseases are separated into the first class or the second class based on at least the PHIs.

18. The system of claim 15, wherein the diseases in the first class add a weight corresponding to a response for a question asked by another disease.

19. The system of claim 15, wherein a sequence of questions corresponds to one of a plurality of evaluation strategies.

20. The system of claim 16, wherein the separation of the diseases into the first class and the second class is dynamic and is based in part on a voting strength of each disease in the list of candidate diseases.

21. The system of claim 20, wherein the voting strength of a particular disease is related to the changing probability that the particular disease is the diagnosis for the patient.

22. The system of claim 20, wherein the voting strength of a particular disease is dependent upon the number of PHIs the patient has of the particular disease.

23. The system of claim 20, wherein the voting strength depends upon aspects of the PHI being established for the patient.

24. The system of claim 20, wherein a particular disease is dynamically transferred between the first and second classes upon reaching or exceeding a threshold.

25. The system of claim 15, wherein the system does not permit diseases of the class not allowed to vote to suggest questions to ask of the patient during an evaluation session based at least in part on the disease score.

26. The system of claim 15, wherein each candidate disease is associated with one or more patient health items (PHIs), and each PHI is associated with one or more questions.

27. The system of claim 16, wherein the computing device additionally performs software instructions to check the patient electronic medical record for responses to questions or PHIs prior to asking questions of the patient.

28. A computerized medical diagnostic system, the system comprising:
a computer storage storing a list of disease objects, each disease object associated with one or more questions; and
a computing device in data communication with the computer storage, the computing device executing instructions associated with
an arbiter object, wherein the arbiter object, in conjunction with a plurality of evaluation strategies, determines the selection of a next best question to ask of a patient.

29. The system of claim 28, wherein the arbiter object determines when the next evaluation strategy of the plurality of evaluation strategies is to be started.

30. The system of claim 29, wherein the determination of when the next evaluation strategy is to be started is based on a rule set.

31. The system of claim 29, wherein the determination of when the next evaluation strategy is to be started depends on the completion of the current evaluation strategy.

32. The system of claim 28, additionally comprising a patient ombudsman object that interfaces with the arbiter object and suggests one or more general questions, wherein answers to the general questions causes a decrease in the number of questions asked of the patient.

33. The system of claim 28, wherein an evaluation strategy is intent modulation in which the late stage of urgent diseases are established or ruled out before proceeding to other diseases.

34. The system of claim 28, wherein an evaluation strategy is intent modulation in which critical curve patient health items (PHIs) of urgent diseases are established after evaluating late stage symptoms.

35. The system of claim 28, wherein an evaluation strategy comprises excluding or establishing serious diseases before diagnosing other diseases.

36. The system of claim 28, wherein the evaluation strategies can be changed as often as after every question asked of the patient.

37. The system of claim 28, wherein certain PHIs of a disease are designated as late stage PHIs of a disease.

38. The system of claim 28, wherein certain PHIs of a disease are designated as critical curve PHIs.

39. The system of claim 28, wherein the arbiter object does not permit disease objects of a class not allowed to vote from the list of disease objects to suggest questions to ask of the patient during an evaluation session.

40. The system of claim 28, wherein each disease object is associated with one or more PHIs, and each PHI is associated with one or more questions.

41. The system of claim 28, wherein the arbiter object interfaces the disease objects with the patient.

42. The system of claim 28, wherein selected ones of the disease objects suggest questions to ask of the patient, and wherein the arbiter object selects the next best question to ask the patient based at least on a voting strength of the selected disease objects.

43. The system of claim 42, wherein the arbiter object selects the next best question to ask the patient additionally based at least on a weight of the question, a sine status of a patient health item associated with the question, the diseases in diagnostic consideration, and data in an electronic medical record of the patient.

44. The system of claim 28, additionally comprising an interface in data communication with an output device to ask questions of the patient and with an input device to receive responses from the patient.

45. The system of claim 28, wherein the arbiter object can change the axis of inquiry based on certain criteria.

46. A computerized arbiter method associated with an evaluation session in a medical diagnostic system, the method comprising:
providing a plurality of modes of inquiry, each mode including at least one evaluation strategy, including one mode of inquiry wherein a plurality of disease objects are separated into a first class that is allowed to vote for the next best question which is to be asked of a patient or into a second class that is not allowed to vote for the next best question to be asked; and
asking the next best question of the patient.

47. The method of claim 46, additionally comprising:
asking general questions of the patient associated with a list of diseases using a high level mode of inquiry;
selecting a set of most likely diseases based on the responses to the general questions;
asking questions focused on the set of most likely diseases using a middle level mode of inquiry;
selecting a most likely disease based on the responses to the questions from the middle level of inquiry; and
asking questions focused on the most likely disease using a low level mode of inquiry,
wherein the evaluation strategies include at least one diagnostic strategy, wherein each of the diseases that has not been excluded from diagnostic consideration adds a weight to a disease score based on a response to each question asked, and wherein questions are asked until a goal of the evaluation session has been reached.

48. The method of claim 46, wherein the evaluation strategies include a non-diagnostic strategy.

49. The method of claim 47, wherein the method does not permit diseases of a non-voting class from the set of most likely diseases to suggest questions to ask of the patient during the evaluation session.

50. The method of claim 47, wherein a disease is excluded if weights for the patient health items for which questions have not been asked yet and weights with associated synergies cannot cause the disease score to reach or exceed a diagnostic threshold.

51. A computerized medical diagnostic system, the system comprising:
a computer storage storing a list of candidate disease objects, each disease object associated with one or more questions; and
a computing device in data communication with the computer storage, the computing device executing instructions associated with
an arbiter object, wherein the arbiter object utilizes at least one of a plurality of evaluation strategies that help determine the selection of a next best question to ask of a patient, wherein the disease objects are separated into a first class that is allowed to vote for the next best question which is to be asked of the patient or into a second class that is not allowed to vote for the next best question.

52. The system of claim 51, wherein the arbiter object determines when a next evaluation strategy of the plurality of evaluation strategies is to be started.
